(19) United States
(12) Patent Application Publication
(10) Pub. No.: US 2024/0269146 A1
(43) Pub. Date: Aug. 15, 2024

(54) USE OF PELABRESIB FOR TREATING ANEMIAS

(71) Applicant: Constellation Pharmaceuticals, Inc., Boston, MA (US)

(72) Inventor: Gözde Colak, Watertown, MA (US)

(21) Appl. No.: 18/290,181

(22) PCT Filed: May 10, 2022
(86) PCT No.: PCT/US2022/028457

§ 371 Date: Nov. 10, 2023

Related U.S. Application Data
(60) Provisional application No. 63/186,978, filed on May 11, 2021.

(51) Int. Cl. A61K 31/55; A61K 31/519; A61P 7/06

(52) U.S. Cl. CPC A61K 31/55; A61K 31/519; A61P 7/06

(57) ABSTRACT

The present disclosure relates to the use of pelabresib, and pharmaceutically acceptable salts thereof, for treating conditions associated with low reticulocyte counts.

### RELATED APPLICATIONS

This application claims priority to U.S. Provisional Application No. 63/186,978, filed May 11, 2021, the entire contents of which are incorporated herein by reference.

### BACKGROUND

Anemia is one of the more common blood disorders affecting approximately 25% of the population or 1.6 billion people worldwide. Anemia occurs when the body has a lower than normal level of healthy red blood cells (RBCs) or when the hemoglobin concentration within them is lower than normal. Hemoglobin enables the RBCs to carry oxygen to the body's tissues. Thus, if there is not enough RBCs, or if the blood cells are abnormal or there is not enough hemoglobin, there will be a decreased capacity in the ability of the blood to transport oxygen to the target tissues. This results in symptoms such as fatigue, weakness, dizziness, shortness of breath, chest pain, and headaches. If left untreated, anemia can lead to severe fatigue (in which a person would not be able to complete everyday tasks), pregnancy complications, heart problems such as enlarged heart or heart failure, and in the most severe instances, death.

RBCs are produced in the bone marrow, where hematopoietic stems cells differentiate and develop, eventually forming reticulocytes. Reticulocytes are immature RBCs and the number of reticulocytes is a good indicator of bone marrow activity because it represents recent production and allows for the determination of reticulocyte count and the reticulocyte production index. These values can be used to determine whether a production problem is contributing to the anemia and can also be used to monitor the progress of treatment for anemia. In some instances, anemias are either hypoproliferative (low reticulocyte count) or hyperproliferative (high reticulocyte count). Hypoproliferative anemias are typically seen when bone marrow is unable to produce adequate red cells. Hyperproliferative anemias involve shortened red cell survival or blood loss.

### SUMMARY

2-((4S)-6-(4-chlorophenyl)-1-methyl-4H-benzo[c]isoxazolo[4,5-e]azepin-4-yl)acetamide, used interchangeably herein with the term pelabresib, is exemplified as Compound 144 in U.S. Pat. No. 8,796,261, and has the following structural formula:

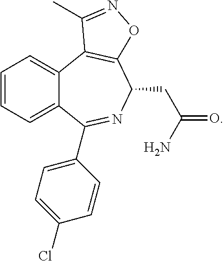 [SMILES: Cc1noc2c1-c1ccccc1C(c1ccc(Cl)cc1)=N[C@H]2CC(N)=O]

Crystalline forms of pelabresib such as the Form A monohydrate are disclosed in U.S. Pat. No. 9,969,747, and, in one aspect, are included as part of the invention. Pelabresib is a potent and selective small molecule designed to promote anti-tumor activity by selectively inhibiting the function of BET protein. See e.g., J. Med. Chem., 2016; Feb. 25; 59(4): 1330-9. The Form A monohydrate of pelabresib is undergoing investigation as both a monotherapy and in combination with the JAK inhibitor ruxolitinib for treating myelofibrosis and related conditions. See e.g., U.S. Clinical Trials NCT02158858 and NCT04603495, and WO 2020/112939.

It has now been found that pelabresib increases the number of reticulocytes in human subjects. See e.g., FIG. 1.

Provided herein, therefore, are methods of using pelabresib, or a pharmaceutically acceptable salt thereof, to treat anemias, particularly those characterized by low reticulocyte count.

Also provided herein are methods of using pelabresib, or a pharmaceutically acceptable salt thereof, to increase the reticulocyte count in subjects in need thereof.

Further provided are methods of using pelabresib, or a pharmaceutically acceptable salt thereof, in combination with a JAK inhibitor such as ruxolitinib, to increase the number of reticulocytes in subjects in need thereof or to treat anemias, particularly those characterized by low reticulocyte count.

### BRIEF DESCRIPTION OF THE FIGURES

FIG. 1 shows the effects of pelabresib on reticulocyte count in subjects with myelofibrosis.

### DETAILED DESCRIPTION

In one embodiment, provided herein is a method of treating an anemia characterized by a low reticulocyte count in a subject in need thereof comprising administering to the subject a therapeutically effective amount of pelabresib, or a pharmaceutically acceptable salt thereof. Also provided is the use of pelabresib, or a pharmaceutically acceptable salt thereof, in the manufacture of a medicament for treating an anemia characterized by a low reticulocyte count in a subject. Further provided is pelabresib, or a pharmaceutically acceptable salt thereof, for treating an anemia characterized by a low reticulocyte count in a subject.

In another embodiment, provided herein is a method of increasing reticulocytes in a subject in need thereof comprising administering to the subject a therapeutically effective amount of pelabresib, or a pharmaceutically acceptable salt thereof. Also provided is the use of pelabresib, or a pharmaceutically acceptable salt thereof, in the manufacture of a medicament for increasing reticulocytes in a subject in need thereof. Further provided is pelabresib, or a pharmaceutically acceptable salt thereof, for increasing reticulocytes in a subject in need thereof.

The terms “subject” and “patient” may be used interchangeably, and mean a mammal in need of treatment, e.g., companion animals (e.g., dogs, cats, and the like), farm animals (e.g., cows, pigs, horses, sheep, goats and the like) and laboratory animals (e.g., rats, mice, guinea pigs and the like). Typically, the subject is a human in need of treatment.

In one aspect, a subject being treated by one of more of the disclosed methods may have myelofibrosis.

The terms “treatment,” “treat.” and “treating” refer to reversing, alleviating, reducing the likelihood of developing, or inhibiting the progress of a disclosed condition (e.g. anemia), or one or more symptoms thereof, as described herein. In some embodiments, treatment may be administered after one or more symptoms have developed, i.e., therapeutic treatment. In other embodiments, treatment may be administered in the absence of symptoms. For example, treatment may be administered to a susceptible individual prior to the onset of symptoms (e.g., in light of a history of symptoms and/or in light of genetic or other susceptibility factors), (i.e., prophylactic treatment). Treatment may also be continued after symptoms have resolved, for example to prevent or delay their recurrence.

Pelabresib may be administered alone, e.g., as a monotherapy or in combination with other active pharmaceutical ingredients (APIs). In one aspect, the other API is a janus kinase (JAK) inhibitor such as ruxolitinib.

As used herein, ruxolitinib refers to the JAK inhibitor (R)-3-(4-(7H-pyrrolo[2,3-d]pyrimidin-4-yl)-1H-pyrazol-1-yl)-3-cyclopentylpropanenitrile phosphate having the following formula:

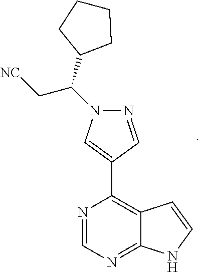 [SMILES: N#CC[C@H](C1CCCC1)n1cc(-c2ncnc3[nH]ccc23)cn1]

As used herein reticulocyte count, as an absolute number or percentage, is a reflection of recent bone marrow activity and can be measured by means known in the art. A normal reticulocyte count, i.e., one that is not low or high, is typically in the range of about 0.5% to about 1.5% of total erythrocytes in the subject. In one aspect, a low reticulocyte count is less than about 0.5% total erythrocytes in the subject.

In one aspect, the anemia characterized by a low reticulocyte count is selected from anemia of chronic renal failure, underproduction anemia, aplastic anemia, iron deficiency anemia, and anemia of inflammation.

In one aspect, the subject being treated is transfusion dependent. In another aspect, the subject being treated is transfusion independent.

The term “effective amount” or “therapeutically effective amount” are used interchangeably and include an amount of a compound described herein that will elicit a desired medical response in a subject, e.g., reducing the symptoms of and/or slowing the progression of the disease.

Pelabresib or the salts and other APIs described herein can be formulated as pharmaceutical compositions and administered to a subject, such as a human, in a variety of forms adapted to the chosen route of administration. Typical routes of administering such pharmaceutical compositions include, without limitation, oral, topical, buccal, transdermal, inhalation, parenteral, sublingual, rectal, vaginal, and intranasal. The term parenteral as used herein includes subcutaneous injections, intravenous, intramuscular, intrathecal, intrasternal injection or infusion techniques. Methods of formulating pharmaceutical compositions are well known in the art, for example, as disclosed in “Remington: The Science and Practice of Pharmacy,” University of the Sciences in Philadelphia, ed., 21st edition, 2005, Lippincott, Williams & Wilkins, Philadelphia, PA.

A specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound described herein in the composition will also depend upon the particular compound in the composition. In one aspect, however, when used as a monotherapy (i.e., without a JAK inhibitor such as ruxolitinib) pelabresib, or a pharmaceutically acceptable salt thereof, may be formulated at a dose of from 50 mg to 500 mg for e.g., administration once, twice, or three times daily. For example, in monotherapies, pelabresib may be administered at a dosage of from 50 mg to 300 mg/day, from 75 mg to 300 mg/day, from 100 mg to 300 mg/day, from 150 mg to 250 mg/day, or at 150 mg/day, 175 mg/day, 200 mg/day, 225 mg/day, or 250 mg/day. In other aspects, when used in combination with a JAK inhibitor such as ruxolitinib, pelabresib, or a pharmaceutically acceptable salt thereof, may be formulated at a dose of from 50 mg to 500 mg for e.g., administration once, twice, or three times daily. For example, in combination therapies, pelabresib may be administered at a dosage of from 50 mg to 300 mg/day, from 75 mg to 300 mg/day, from 100 mg to 300 mg/day, from 100 mg to 200 mg/day, or at 100 mg/day, 125 mg/day, 150 mg/day, 175 mg/day, or 200 mg/day.

### EXEMPLIFICATION

Pelabresib and the Form A monohydrate can be obtained following the procedures described in U.S. Pat. Nos. 8,796,261 and 9,969,747 respectively.

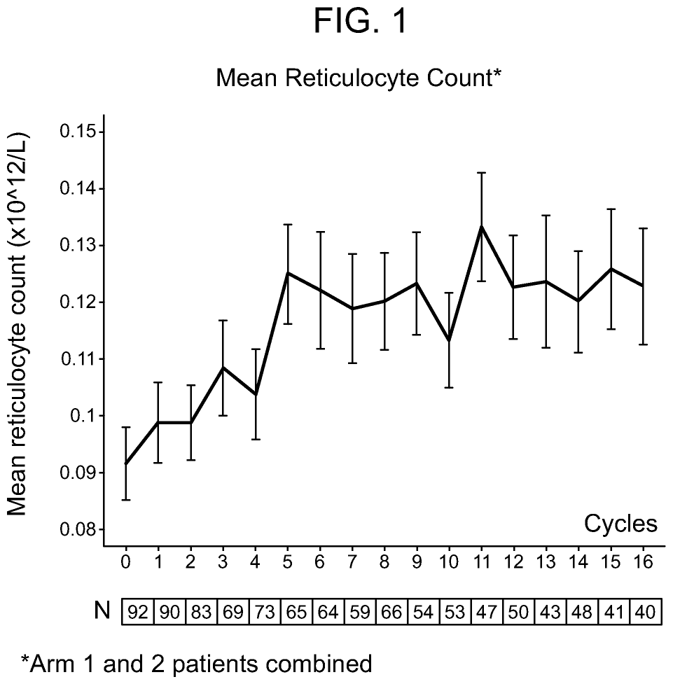

Pelabresib Form A monohydrate was administered to human subjects (with or without ruxolitinib) with a median starting dose of 125 mg QD and a max dose of 225 mg QD for a median duration of 47 weeks. The results of this study are shown in FIG. 1 (Arm 1 being pelabresib Form A and Arm 2 being pelabresib Form A in combination with ruxolitinib). As shown, pelabresib was effective in increasing reticulocytes and improving hemoglobin.

While have described a number of embodiments of this, it is apparent that our basic examples may be altered to provide other embodiments that utilize the compounds and methods of this disclosure. Therefore, it will be appreciated that the scope of this disclosure is to be defined by the appended claims rather than by the specific embodiments that have been represented by way of example.

The contents of all references (including literature references, issued patents, published patent applications, and co-pending patent applications) cited throughout this application are hereby expressly incorporated herein in their entireties by reference. Unless otherwise defined, all technical and scientific terms used herein are accorded the meaning commonly known to one with ordinary skill in the art.

1. A method of treating an anemia characterized by a low reticulocyte count in a subject in need thereof comprising administering to the subject a therapeutically effective amount of pelabresib, or a pharmaceutically acceptable salt thereof.

2. The method of claim 1, wherein the low reticulocyte count is less than about 0.5% of total erythrocytes in the subject.

3. The method of claim 1 or 2, wherein the subject has myelofibrosis.

4. The method of any one of claims 1 to 3, wherein the anemia is selected from anemia of chronic renal failure, underproduction anemia, aplastic anemia, iron deficiency anemia, and anemia of inflammation.

5. A method of increasing reticulocytes in a subject in need thereof, comprising administering to the subject a therapeutically effective amount of pelabresib, or a pharmaceutically acceptable salt thereof.

6. The method of claim 5, wherein the subject has a reticulocyte count of less than about 0.5% of total erythrocytes in the subject prior to administering pelabresib, or a pharmaceutically acceptable salt thereof.

7. The method of claim 5 or 6, wherein the subject has anemia.

8. The method of any one of claims 5 to 7, wherein the subject has myelofibrosis.

9. The method of any one of claims 1 to 8, further comprising administering to the subject a therapeutically effective amount of a janus kinase (JAK) inhibitor.

10. The method of claim 9, wherein the JAK inhibitor is ruxolitinib.

11. The method of any one of claims 1 to 10, wherein the pelabresib is Form A monohydrate.
